SEQUENCE LISTING (8 sequences)

SEQ ID NO: 1 - DNA, 1433 bp, Homo sapiens
agatgccccacagcactgctcttccagaggcaagaccaaccaagatgagtgccctgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtatacaccttaaatgataagaagcagtggataaataaggctgttggagataaacttcctgaatgtgaagcagatgacggctgcccgaagccccccgagattgcacatggctatgtggagcactcggttcgctaccagtgtaagaactactacaaactgcgcacagaaggagatggagtgtacaccttaaacaatgagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccattt

SEQ ID NO: 2 - DNA, 35 bp, Homo sapiens
cgggatccatgacagctgcccaaagcccccagaga

SEQ ID NO: 3 - DNA, 43 bp, Homo sapiens
cggaattccagctgtcatctgcctcacattcggggagtttctc

SEQ ID NO: 4 - DNA, 21 bp, Mus musculus
agccctgggagctgttgtcac

SEQ ID NO: 5 - DNA, 22 bp, Mus musculus
tgggtgctccgatggctctctg

SEQ ID NO: 6 - DNA, 20 bp, Mus musculus
gcctagccgagggagagccg

SEQ ID NO: 7 - DNA, 22 bp, Mus musculus
tgtgacttgggagctctgcagc

SEQ ID NO: 8 - DNA, 19 bp, Mus musculus
gcccgccccgactgcatct

(19) United States
(12) Patent Application Publication
(10) Pub. No.: US 2007/0101445 A1
(43) Pub. Date: May 3, 2007

(54) Transgenic mice carrying the HP-2 gene and uses as models for vascular diseases

(76) Inventors: Andrew Levy, Kiryat Shmuel (IL); Nina Levy, Kiryat Shmuel (IL)

Correspondence Address: PEARL COHEN ZEDEK LATZER, LLP, 1500 BROADWAY 12TH FLOOR, NEW YORK, NY, 10036, US

(21) Appl. No.: 11/584,762

(22) Filed: Oct. 23, 2006

Related U.S. Application Data
(63) Continuation-in-part of application No. PCT/US06/26842, filed on Jul. 12, 2006.
(60) Provisional application No. 60/698,012, filed on Jul. 12, 2005.

(51) Int. Cl. A01K 67/027; C12N 5/06

(52) U.S. Cl. 800/3; 800/18; 435/354

(57) ABSTRACT

This invention provides transgenic mice carrying the humanized Hp-2 allele for haptoglobin. Specifically, provided herein are methods of the use of these transgenic mice in the diagnosis and rational drug design of compounds to be used in the treatment of vascular complications in diabetic subjects carrying the Hp-2 gene.

### CROSS-REFERENCE TO RELATED APPLICATIONS

This application is a continuation-in-part of application Ser. No. PCT/US2006/026842, filed Jul. 12, 2006, which claims priority from U.S. provisional application Ser. No. 60/698,012, filed Jul. 12, 2005, both of which are incorporated by reference herein in their entireties.

### FIELD OF INVENTION

This invention relates to transgenic mice carrying the humanized Hp-2 allele for haptoglobin. Specifically, the invention relates to the use of these transgenic mice in methods of diagnosis and rational drug design for compounds to be used in the treatment of macrovascular and microvascular complications, including atherosclerosis and diabetic complications, in human subjects.

### BACKGROUND OF THE INVENTION

The major cause of acute coronary thrombosis is atherosclerotic plaque rupture and the precursor lesion has been termed the high-risk plaque (Burke A P, Farb A, Malcolm G T, Liang Y H, Smialek J, Virmani R. Coronary risk factors and plaque morphology in men with coronary artery disease who die suddenly. N Engl J Med. 1997;336:1276-1282; Virmani R, Kolodgie F D, Burke A P, Farb A, Schwartz S M. Lessons from sudden coronary death: a comprehensive morphological classification scheme for atherosclerotic lesions. Arterioscl Thromb Vasc Biol. 2000;20: 1262-1275; Fuster V, Moreno P R, Fayad Z A, Corti R, Badimon J J. Atherothrombosis and the high-risk plaque. Part I. J Am Coll Card. 2005;46: 937-944; Fuster V, Fayad Z A, Moreno P R, Poon M, Corti R, Badimon J J. Atherothrombosis and the high-risk plaque. Part II. J Am Coll Card. 2005;46: 1209-1218; Kolodgie F D, Gold H K, Burke A P, Fowler D R, Kruth H S, Weber D K, Farb A, Guerrero L J, Hayase M, Kutys R, Narula J, Finn A V, Virmani R. Intraplaque hemorrhage and progression of coronary atheroma. N Eng J Med. 2003;349:2316-2325; Virmani R, Kolodgie F D, Burke A P, Finn A V, Gold H K, Tulenko T N, Wrenn S P, Narula J. Atherosclerotic plaque progression and vulnerability to rupture angiogenesis as a source of intraplaque hemorrhage. Arterioscl Thromb Vasc Biol. 2005;25:2054-2061). Pathological features of high risk plaques include a large lipid necrotic core, thin fibrous cap, inflammatory infiltrate and intraplaque hemorrhage. Extracorpuscular hemoglobin (Hb) released from red blood cells after intra-plaque hemorrhage represents a potent stimulus for inflammation within the plaque. It is becoming apparent that the frequency of microvascular hemorrhages has been severely underestimated and may occur in up to 40% of all advanced atherosclerotic plaques (Kockx M M, Cromheeke K M, Knaapen M W M, Bosmans J M, De Meyer G R Y, Herman A G, Bult H. Phagocytosis and macrophage activation associated with hemorrhagic microvessels in human atherosclerosis. Arterioscl Thromb Vasc Biol. 2003;23:440-446).

An important defense mechanism to counteract the effects of intra-plaque hemorrhage is mediated by haptoglobin (Hp), an abundant serum protein whose primary function is to bind to extracorpuscular hemoglobin (Hb) thereby attenuating its oxidative and inflammatory potential (Bowman B H, Kurosky A. Haptoglobin: the evolutionary product of duplication, unequal crossing over, and point mutation. Adv Hum Genet. 1982;12:189-261.). Hp also promotes the clearance of extracorpuscular Hb via the CD163 scavenger receptor present on macrophages (Kristiansen M, Graversen J H, Jacobsen C, Sonne O, Hoffman H J, Law S K, Moestrup S K. Identification of the hemoglobin scavenger receptor. Nature. 2001;409:198-201). This scavenging pathway is the only mechanism that exists for removing free Hb released at extravascular sites, i.e. at sites of hemorrhage within the atherosclerotic plaque.

In man there exists two classes of alleles for Hp, designated 1 and 2. The Hp polymorphism is a common polymorphism. In the western world, 16% of the population is Hp 1-1 (homozygous for the Hp 1 allele), 36% is Hp 2-2 (homozygous for the Hp 2 allele) and 48% is Hp 2-1 (heterozygote) (Bowman B H, Kurosky A, op. cit.). The Hp 2 allele is found only in man. All other mammals, including higher primates have only the Hp 1 allele and therefore have the Hp 1-1 genotype. The Hp 2 allele appears to have been generated by an intragenic duplication event of exons 3 and 4 of the Hp 1 allele approximately 100,000 years ago early in human evolution (Bowman B H, Kurosky A, op. cit.).

In multiple independent longitudinal and cross sectional studies from diverse ethnic groups and geographic areas, it has been demonstrated that the Hp 2-2 genotype is associated with an increased risk of atherosclerotic cardiovascular disease and its sequelae such as acute myocardial infarction (Langlois M R, Delanghe J R. Biological and clinical significance of haptoglobin polymorphism in humans. Clin Chem 1996;42:1589-1600; Levy A P, Hochberg I, Jablonski K, Resnick H, Best L, Lee E T, Howard B V. Haptoglobin phenotype and the risk of cardiovascular disease in individuals with diabetes: The Strong Heart Study. J Am Coll Card. 2002; 40:1984-1990; Roguin A, Koch W, Kastrati A, Aronson D, Schomig A, Levy A P. Haptoglobin genotype is predictive of major adverse cardiac events in the one year period after PTCA in individuals with diabetes. Diabetes Care. 2003;26:2628-2631; Suleiman M, Aronson D, Asleh R, Kapelovich M R, Roguin A, Meisel S R, Shochat M, Suleiman A, Reisner S A, Markiewicz W, Hammerman H, Lotan R, Levy N S, Levy A P. Haptoglobin polymorphism predicts 30-day mortality and heart failure in patients with diabetes and acute myocardial infarction. Diabetes. 2005;19:2802-2806). In vitro fundamental differences in the antioxidant and immunomodulatory properties of the Hp 1-1 and Hp 2-2 proteins may explain why Hp is a susceptibility gene for cardiovascular disease (CVD). As an antioxidant the Hp 1-1 protein is superior to the Hp 2-2 protein in blocking the oxidative action of Hb (Frank M, Lache O, Enav B, Szafranek T, Levy N S, Ricklis R M, Levy A P. Structure/function analysis of the anti-oxidant properties of haptoglobin. Blood. 2001;98:3693-3698; Asleh R, Guetta J, Kalet-Litman S, Miller-Lotan R, Levy A P. Haptoglobin genotype and diabetes dependent differences in iron mediated oxidative stress in vitro and in vivo. Circ Res. 2005;96:435-441; Asleh R, Marsh S, Shiltruck M, Binah O, Guetta J, Lejbkowicz F, Enav B, Shehadeh N, Kanter Y, Lache O, Cohen O, Levy N S, Levy A P. Genetically determined heterogeneity in hemoglobin scavenging and susceptibility to diabetic cardiovascular disease. Circ Res. 2003;92:1193-1200). As an immunomodulator, the Hp 1-1-Hb complex stimulates the macrophage to secrete anti-inflammatory cytokines to a markedly greater degree than the Hp 2-2-Hb complex (Philippidis P, Mason J C, Evans B J, Nadra I, Taylor K M, Haskard D O, Landis R C. Hemoglobin scavenger receptor CD163 mediates interleukin 10 release and heme oxygenase-1 synthesis: anti-inflammatory monocyte-macrophage responses in vitro, in resolving skin blisters in vivo, and after cardiopulmonary bypass surgery. Circ Res. 2004;94:119-126; Philippidis P, Boyle J J, Domin J, Nadra I, Haskard D O, Taylor K M. Anti-inflammatory hemoglobin scavenging macrophages in atherosclerotic plaques: a potential atheroprotective role. Circ. 2005;112:431(abstract); Guetta J, Strauss M, Levy N S, Fahoum L, Levy A P. Haptoglobin genotype modulates the balance of Th1/Th2 cytokines produced by macrophages exposed to free hemoglobin. Atherosclerosis. 2006; Jul 1 Epub).

An experimental model could be used to screen for agents that inhibit, prevent, or reverse the progression of macrovascular and microvascular complications, including atherosclerosis as well as diabetes mellitus (DM)-related vascular complications. Such models could be employed to develop pharmaceuticals that are effective in preventing, arresting or reversing vascular disease. Only humans develop any of the pathological features of DM-related vascular complications associated with the Hp-2 gene. The expense and difficulty of using primates and the length of time required for developing the DM-related pathology of vascular complications makes extensive research on such animals prohibitive. Rodents do not develop DM-related vascular complications associated with the Hp-2 gene.

### SUMMARY OF THE INVENTION

In one embodiment provided herein is a transgenic mouse whose genome comprises a nucleic acid encoding a humanized Hp-2 gene, wherein said humanized Hp 2 gene comprises the extracellular domain of a human Hp-2 gene, and said nucleic acid comprises exons 5 and 6 of a human Hp-2 gene, and exons 1,2 3, 4 and of a mouse or human Hp-1 gene (see FIG. 1).

In one embodiment, provided herein is a transgenic mouse whose genome comprises a nucleic acid which does not encode murine Hp gene.

In another embodiment, provided herein is a method for identifying in vivo a biological activity of a compound, said method comprising the steps of: providing a transgenic mouse expressing humanized Hp-2 gene; administering said compound to said mouse; determining an expressed pathology of said mouse; and identifying a in vivo biological activity of said compound. In another embodiment, the transgenic mouse is diabetic. In another embodiment, diabetes is induced by administration of streptozotocin.

In one embodiment, provided herein is a method for evaluating in a transgenic mouse the potential therapeutic effect of a compound for treating pathogenesis of a vascular disease in a human, which comprises: administering the compound to the transgenic mouse embodied herein, wherein said mouse exhibits at least one vascular disease which is atherosclerosis, myocardial infarct, cardiovascular disease, cerebrovascular disease, a complication of diabetes, nephropathy, retinopathy, or neuropathy; and determining the therapeutic effect of the compound on the transgenic mouse. In another embodiment, the transgenic mouse is diabetic. In another embodiment, diabetes is induced by administration of streptozotocin. In another embodiment, the mouse exhibits increased iron deposition in plaque, increased lipid peroxidation in plaque, increased ceroid in plaque, or increased macrophage accumulation in plaque.

In another embodiment, provided herein is a method of making a transgenic mouse comprising: introducing into a mouse embryo a polynucleotide comprising a coding region which encodes Hp-2 gene product; transferring the embryo into a foster mother mouse; permitting the embryo to gestate; and selecting a transgenic mouse born to said foster mother mouse, wherein said transgenic mouse is characterized in that it has an increased probability of developing atherosclerosis, including increased iron deposition in plaque, increased lipid peroxidation in plaque, increased ceroid in plaque, increased macrophage accumulation in plaque, or diabetes-related vascular complications, when compared to a non-transgenic littermate.

In one embodiment, provided herein is a method of culturing transgenic cells comprising the steps of: providing a cell taken from a transgenic mouse of the invention; and culturing said cell under conditions that allow growth of said cell.

### BRIEF DESCRIPTION OF THE DRAWINGS

FIG. 1A-D depict the construction of a murine Hp 2 allele. FIG. 1A shows a schematic diagram of the exon structure of the Hp gene (1 or 2 allele). FIG. 1B also shows the Hp 1 and Hp2 exon structures and the structure of the murine Hp 2 described herein. FIG. 1C shows a fine map of the murine Hp locus before and after gene targeting. At the top, the genomic organization of the murine Hp 1 allele is shown, including B, Bam H1; Bg, Bgl II; E, EcoR1; and P, PvuII sites. In the middle, the genomic organization of the murine Hp 2 allele is shown after successful gene targeting by homologous recombination. At the bottom is shown the genomic organization of the murine Hp 2 allele after removal of the Neo and CD cassettes with cre recombinase. FIG. 1D shows a Southern blot of ES transfectants with successful gene targeting, demonstrating an additional band of 11 kb recognized by the probe;

FIG. 2A-B show that the size and shape of murine Hp 2 polymers are similar to human Hp 2 polymers. In FIG. 2A, a schematic illustration shows the shapes of Hp polymers in humans with the Hp 1-1, Hp 2-1 or Hp 2-2 genotypes. FIG. 2B demonstrates that the polymer distribution in murine Hp 1-1, 2-1 and 2-2 mice is similar to that in humans with Hp 1-1, 2-1 and 2-2;

FIG. 3 shows increased iron in plaques from Hp 2-2 mice (right panel), versus Hp 1-1 mice (left panel);

FIG. 4A-B show increased lipid peroxidation (FIG. 4A) and ceroid (FIG. 4B) in plaques of Hp 2-2 mice (right panels), compared to Hp 1-1 mice (left panels); and

FIG. 5A-D show increased macrophage accumulation in the plaques of Hp 2-2 mice. In FIG. 5A and 5B, representative plaques are shown of similar size but with dramatically greater macrophage accumulation in Hp 2-2 Apo E−/− (A) as compared to Hp 1-1 ApoE −/− (B) mice. FIG. 5C shows a histogram of the mean±SEM of the number of macrophages in the intima and adventitia from all plaques (n=18 for Hp 1-1 and n=15 for Hp 2-2). FIG. 5D plots the number of intimal macrophages vs. the lipid core area (μm²) in plaques from Hp 1-1 ApoE−/− (n=18) and Hp 2-2 ApoE−/− (n=15) mice.

### DETAILED DESCRIPTION OF THE INVENTION

Unless otherwise specified, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

In one embodiment, mice transgenic for the human Hp 2 allele are provided for the evaluation of agents that can prevent or intervene in the development of atherosclerosis and other vasculopathies resulting from the presence of the Hp 2 gene, which increases susceptibility to oxidant stress. The model is also useful in other embodiments for studying the development of vasculopathies in diabetes, both in macrovascular diseases (cardiovascular and cerebrovascular diseases) as well as microvascular diseases (retinopathy, nephropathy and neuropathy) because diabetic patients are at increased risk for such complications.

Intraplaque hemorrhage increases the risk of plaque rupture and thrombosis. The release of hemoglobin (Hb) from extravasated erythrocytes at the site of hemorrhage leads to iron deposition, which may increase oxidation and inflammation in the atherosclerotic plaque. The haptoglobin (Hp) protein is critical for protection against Hb-induced injury. In humans, the Hp genotype confers dramatic differences in susceptibility to developing diabetic vascular complications. In one embodiment the association between Hp genotype and diabetic vascular disease are validated by identifying Hp genotype and diabetes-dependent differences in renal pathology in mice, genetically modified at the Hp gene locus. Two common alleles exist at the Hp locus and the Hp 2 allele has been associated with increased risk of myocardial infarction. The Hp 2 protein provides decreased anti-oxidative and anti-inflammatory activity. As will be shown in the examples below, the Hp 2-2 genotype is associated with increased iron deposition, lipid peroxidation product, ceroid deposition and macrophage accumulation in atherosclerotic plaques.

In another embodiment, the model described herein is used to provide direct evidence that the Hp genotype contributes to the modulation of the number of macrophages in the atherosclerotic plaque. There is significantly greater macrophage accumulation in the intima and adventitia of atherosclerotic plaques of Hp 2-2 as compared to Hp 1-1 mice. Moreover, there is increased iron deposition, accumulation of lipid peroxidation products and ceroid in plaques in Hp 2-2 mice. Data collected from the model provide a framework linking intraplaque microvascular hemorrhage, the size of the necrotic lipid core and inflammation in determining plaque vulnerability.

In another embodiment, the model is useful for evaluating effects of preventionary or interventionary maneuvers on those features of plaque, including but not limited to increased iron deposition, increased lipid peroxidation, increased ceroid accumulation and increased macrophage accumulation in atherosclerotic plaque. In another embodiment, the model is studied in the setting of diabetes, in which in addition to accelerated macrovascular complications seen in comparison to that in non-diabetic animals, microvascular complications also develop, including but not limited to retinopathy, nephropathy (kidney disease) and neuropathy. Diabetes can be induced chemically, such as in one embodiment using streptozotocin, or in other embodiments, induced genetically by introducing one or more appropriate genes into the Hp 2 mouse through breeding or transgenic means.

The diabetes and genotype-dependent morphometric and histological differences described herein are due in another embodiment to a significant increase in iron deposition in the kidneys of the Hp 0 and Hp 2 mice. While iron deposits are significantly increased in both Hp 0 and Hp 2 mice in the presence and absence of diabetes, the amount of iron deposition was found to be significantly increased in diabetes. The potential pathological significance of these iron deposits are in one embodiment, diabetes dependent. In another embodiment, iron-induced oxidation is shown to be glucose dependent and in another embodiment, may be accelerated in the diabetic state due to the ability of glucose to recycle the ferrous (+3) iron to the ferric (+2) state with markedly greater oxidative potential. Iron-mediated damage in diabetic vascular complications has in one embodiment, an important role. Increased proximal tubular iron is observed in another embodiment, in patients with diabetic nephropathy. A synergy between hyperglycemia and iron is proposed for explaining in another embodiment, the accelerated macrovascular disease found in diabetic individuals. Iron chelation therapy is shown to prevent in one embodiment, diabetic vascular complications in several models and in man.

In one embodiment, provided herein is a transgenic mouse whose genome comprises a nucleic acid encoding a humanized Hp-2 gene, wherein said humanized Hp 2 gene comprises the extracellular domain of a human Hp-2 gene, and said nucleic acid comprises exons 5 and 6 of a human Hp-2 gene, and exons 1,2 3, 4 and of a mouse or human Hp-1 gene.

Generally, the nomenclature used herein and the laboratory procedures in cell culture, molecular genetics, nucleic acid chemistry and hybridization, biochemistry, histology and immunocytochemistry described below are those well known and commonly described in the art. Standard techniques are used for recombinant nucleic acid methods, polynucleotide synthesis, cell culture, transgene incorporation, Western blotting, immunocytochemistry and histological techniques such as silver staining. The techniques and procedures are generally performed according to conventional methods in the art and various general references which are provided throughout this specification. The procedures therein are well known in the art and are provided for the convenience of the reader. All the information contained therein is incorporated herein by reference.

Mice are used in one embodiment for transgenic animal models because they are easy to house, relatively inexpensive, and easy to breed. However, other non-human transgenic mammals may also be made in accordance with the present invention and in certain embodiments, such as monkeys, sheep, rabbits or rats. In one embodiment, transgenic animals refer to those animals that carry a transgene, which is a cloned gene introduced and stably incorporated, which is passed on in another embodiment, to successive generations. In an embodiment of the present invention, the humanized Hp-2 gene was cloned and stably incorporated into the genome of a mouse. Alternatively, altered portions of the Hp-2 gene sequence may be used in other embodiments. In this manner, the specific function of alternatively spliced gene products may be investigated during animal development and initiation of malignancy in order to develop therapeutic strategies.

To create a transgenic mouse, an altered version of the human gene of interest is inserted in one embodiment, into a mouse germ line using standard techniques of oocyte microinjection or transfection or microinjection into stem cells. In another embodiment, if it is desired to inactivate or replace the endogenous gene, homologous recombination using embryonic stem cells may be applied.

For oocyte injection, one or more copies of the human Hp-2 gene sequence can be inserted into the pronucleus of a just-fertilized mouse oocyte. This oocyte is then reimplanted into a pseudo-pregnant foster mother. The liveborn mice can then be screened for integrants using analysis of tail DNA for the presence of the Hp-2 gene sequences. The transgene can be either a complete genomic sequence injected as a YAC or chromosome fragment, a cDNA with either the natural promoter or a heterologous promoter, or a minigene containing all of the coding region and other elements found to be necessary for optimum expression.

Retroviral infection of early embryos can also be done to insert the altered gene. In this method, the altered gene is inserted into a retroviral vector which is used to directly infect mouse embryos during the early stages of development to generate a chimera, some of which will lead to germline transmission (Jaenisch, R. 1976. Proc. Natl. Acad. Sci. USA, 73: 1260-1264, which is incorporated herein by reference in its entirety).

In one embodiment, “transfection” refers to a cell that has been “transformed” or “transfected” with exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a vector or plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or ancestor by mitosis. A “cell line” is a clone of a primary or other cell that is capable of stable growth in vitro for many generations. An organism, such as a plant or animal, that has been transformed with exogenous DNA is termed “transgenic”, such as, in one embodiment, the transgenic mouse described herein.

One skilled in the art would readily comprehend that the nucleic acid construct of certain embodiments of the present invention may contain, any suitable nucleic acid sequence which encodes for the Hp-2 gene. Such nucleic acid sequence is in another embodiment, the full-length Hp-2 cDNA or may encompass other variants or derivatives of such sequence so long as the Hp-2 gene is expressed in other embodiments. Nucleic acid variants are those that comprise in one embodiment, a sequence substantially different from the Hp-2 cDNA sequence but that, due to the degeneracy of the genetic code, still encode Hp-2. The variants may be variants made in another embodiment, by recombinant methods such as in one embodiment, mutagenesis techniques. Such nucleic acid variants include in one embodiment, those produced by nucleotide substitutions, deletions or additions. The substitutions, deletions or additions may involve in another embodiment, one or more nucleotides. Alterations in the coding regions may produce in one embodiment, conservative or nonconservative amino acid substitutions, deletions or additions. In one embodiment these substitutions, deletions or additions are silent substitutions, additions and deletions which do not alter the properties and activities of the Hp-2 gene. Nucleotide changes present in a variant polynucleotide are silent in one embodiment, which means in another embodiment, that they do not alter the amino acids encoded by the polynucleotide.

One skilled in the art would also understand that the Hp-2 gene may be obtained by a wide variety of techniques that include, but are not limited to, isolation from genomic sources, preparation of cDNAs from isolated mRNA templates, direct synthesis, or a combination thereof. These techniques are well known to those of skill in the art. Furthermore, the Hp-2 gene has been previously described and characterized and therefore one skilled in the art would readily comprehend what gene and sequence is encompassed by reference to the “Hp-2” gene. The nucleic acid construct of the present invention include in one embodiment, a regulatory element in order to enhance the expression of the Hp-2 transgene.

The following terms are used to describe the sequence relationships between two or more nucleic acid molecules or polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence.

Optimal alignment of sequences for aligning a comparison window may be conducted by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Natl. Acad. Sci. (USA) 85:2444, or by computerized implementations of these algorithms (GAP, BESTFIT, FASTA, and TFASTA in the Wisconsin Genetics Software Package Release 7.0, Genetics Computer Group, 575 Science Dr., Madison, Wis.).

“Substantial identity” or “substantial sequence identity” mean that two peptide sequences, when optimally aligned, such as by the programs GAP or BESTFIT using default gap which share at least 90 percent sequence identity, preferably at least 95 percent sequence identity, more preferably at least 99 percent sequence identity or more. “Percentage amino acid identity” or “percentage amino acid sequence identity” refers to a comparison of the amino acids of two polypeptides which, when optimally aligned, have approximately the designated percentage of the same amino acids. For example, “95% amino acid identity” refers to a comparison of the amino acids of two polypeptides which when optimally aligned have 95% amino acid identity. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

The percent identity between the two sequences is a function of the number of identical positions shared by the sequences (i.e., % identity of identical positions/total # of positions (e.g., overlapping×100). Preferably, the two sequences are the same length. The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci.

USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to Hp-2 nucleic acid molecules of the invention. BLAST protein searches can be performed with the X13LAST program, score=50, wordlength=3 to obtain amino acid sequences homologous to Hp-2 protein molecules of the invention. To obtain gapped alignments for comparison purposes, Gapped BLAST can be utilized as described in Altschul et al., (1997) Nucleic Acids Res. :3389-3402. When utilizing BLAST and Gapped BLAST programs, the default parameters of the respective programs (e.g., X13LAST and NBLAST) can be used. See http://www.ncbi.nlm.nih.gov. Another preferred, non-limiting example of a mathematical algorithm utilized for the comparison of sequences is the algorithm of Myers and Miller, CABIOS 4:11-17 (1988). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, only exact matches are counted.

In one embodiment, also within the scope of the invention, are isolated Hp-2 proteins having an amino acid sequence that is at least about 75%, 85%, 90%, 95%, or 98% identical to the amino acid sequence of Hp-2 as compared with the following sequence:

(SEQ ID NO. 1)

| | |
|---|---|
| 1 | agatgcccca cagcactgct cttccagagg caagaccaac |
| | caagatgagt gccctgggag |
| 61 | ctgtcattgc cctcctgctc tggggacagc tttttgcagt |
| | ggactcaggc aatgatgtca |
| 121 | cggatatcgc agatgacggc tgcccgaagc cccccgagat |
| | tgcacatggc tatgtggagc |
| 181 | actcggttcg ctaccagtgt aagaactact acaaactgcg |
| | cacagaagga gatggagtat |
| 241 | acaccttaaa tgataagaag cagtggataa ataaggctgt |
| | tggagataaa cttcctgaat |
| 301 | gtgaagcaga tgacggctgc ccgaagcccc ccgagattgc |
| | acatggctat gtggagcact |
| 361 | cggttcgcta ccagtgtaag aactactaca aactgcgcac |
| | agaaggagat ggagtgtaca |
| 421 | ccttaaacaa tgagaagcag tggataaata aggctgttgg |
| | agataaactt cctgaatgtg |
| 481 | aagcagtatg tgggaagccc aagaatccgg caaacccagt |
| | gcagcggatc ctgggtggac |
| 541 | acctggatgc caaaggcagc tttccctggc aggctaagat |
| | ggtttcccac cataatctca |
| 601 | ccacaggtgc cacgctgatc aatgaacaat ggctgctgac |
| | cacggctaaa aatctcttcc |
| 661 | tgaaccattc agaaaatgca acagcgaaag acattgcccc |
| | tactttaaca ctctatgtgg |
| 721 | ggaaaaagca gcttgtagag attgagaagg ttgttctaca |
| | ccctaactac tcccaggtag |
| 781 | atattgggct catcaaactc aaacagaagg tgtctgttaa |
| | tgagagagtg atgcccatct |
| 841 | gcctaccttc aaaggattat gcagaagtag ggcgtgtggg |
| | ttatgtttct ggctgggggc |
| 901 | gaaatgccaa ttttaaattt actgaccatc tgaagtatgt |
| | catgctgcct gtggctgacc |
| 961 | aagaccaatg cataaggcat tatgaaggca gcacagtccc |
| | cgaaaagaag acaccgaaga |
| 1021 | gccctgtagg ggtgcagccc atactgaatg aacacacctt |
| | ctgtgctggc atgtctaagt |
| 1081 | accaagaaga cacctgctat ggcgatgcgg gcagtgcctt |
| | tgccgttcac gacctggagg |
| 1141 | aggacacctg gtatgcgact gggatcttaa gctttgataa |
| | gagctgtgct gtggctgagt |
| 1201 | atggtgtgta tgtgaaggtg acttccatcc aggactgggt |
| | tcagaagacc atagctgaga |
| 1261 | actaatgcaa ggctggccgg aagcccttgc ctgaaagcaa |
| | gatttcagcc tggaagaggg |
| 1321 | caaagtggac gggagtggac aggagtggat gcgataagat |
| | gtggtttgaa gctgatgggt |
| 1381 | gccagccctg cattgctgag tcaatcaata aagagctttc |
| | ttttgaccca ttt |

In another embodiment, provided herein is a transgenic mouse whose genome comprises a nucleic acid encoding a humanized Hp-2 gene. In another embodiment, the humanized Hp 2 gene comprises the extracellular domain of a human Hp-2 gene, and said nucleic acid comprises exons 5 and 6 of a human Hp-2 gene, wherein exons 5 and 6 of said human Hp-2 gene are a duplicate of exons 3 and 4 of said mouse or human Hp 1 gene respectively.

In one embodiment, provided herein is a transgenic mouse whose genome comprises a nucleic acid encoding a humanized Hp-2 gene, wherein said transgenic mouse exhibits, relative to a wild-type mouse, an increased sensitivity to vascular damage, such as atherosclerosis in one embodiment, or myocardial infract, cerebrovascular disease, nephropathy, retinopathy, neuropathy or cardiovascular disease in other embodiments. In another embodiment, increased susceptibility to diabetic complications is provided.

In another embodiment, provided herein is a cell obtained from the transgenic mice described herein.

In another embodiment, provided herein is a transgenic mouse whose genome comprises a nucleic acid which does not encode murine Hp gene. In one embodiment, this mouse is referred to is Hp-0 mouse.

A transgenic animal carrying one transgene can be further bred to another transgenic animal carrying a second transgenes to create a so-called “double transgenic” animal carrying two transgenes. In one embodiment the invention relates to non-human transgenic animals having a transgene comprising a polynucleotide sequence encoding a humanized Hp-2 of the invention or in another embodiment, having an additional transgene encoding a gene of interest operably linked to a Hp-2 responsive promoter. In one embodiment, the double transgenic mouse of the invention further comprises a polynucleotide sequence, encoding a gene or in another embodiment, a protein of interest, which in one embodiment encodes a gene encoding a detectible marker or a detectible protein. Double transgenic animals having both transgenes (i.e., a HP-2 transgene and a gene of interest linked to a Hp-2-responsive promoter) are also encompassed by the invention.

In another embodiment, provided herein is a method for identifying in vivo a biological activity of a compound, said method comprising the steps of: providing a transgenic mouse expressing a humanized Hp-2 gene; administering said compound to said mouse; determining an expressed pathology of said mouse; and identifying a in vivo biological activity of said compound. In other embodiments, the pathology can be increased iron deposition in plaque or kidneys, increased lipid peroxidation in plaque, increased ceroid deposition in plaque, increased macrophage accumulation in plaque, increased renal mass, among others.

The compounds referred to can be of any type, including in one embodiment, nucleic acid, polypeptide or other organic molecule including a small molecule. The present invention extends in various aspects to a pharmaceutical composition, medicament, drug or other composition comprising such a compound, a method comprising administration of such a composition comprising such a compound, a method comprising administration of such a composition to a patient, e.g., for treatment of vascular sensitivities and pathologies, use of such a compound in the manufacture of a composition for administration, e.g., for treatment of vascular pathologies, and a method of making a pharmaceutical composition comprising admixing such a compound with a pharmaceutically acceptable excipient, vehicle or carrier, and optionally other ingredients.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like.

The active agent is preferably administered in a therapeutically effective amount. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Alternatively, targeting therapies may be used to deliver the active agent more specifically to certain types of cell, by the use of targeting systems such as antibodies or cell specific ligands. Targeting may be desirable for a variety of reasons, e.g. if the agent is unacceptably toxic, or if it would otherwise require too high a dosage, or if it would not otherwise be able to enter the target cells.

As will be shown in the ensuing examples, prior in vitro studies demonstrating significant differences in the anti-oxidant and anti-inflammatory properties of the Hp 1 and Hp 2 allele gene products, provide a basis to explain the in vivo observations embodied herein. Intra-plaque hemorrhage generates greater iron deposition in mice with the Hp 2-2 genotype, leading to increased oxidation of lipids and other cellular constituents of the plaque. Notably, iron and ceroid have been reported to be colocalized in human atherosclerotic specimens (Lee F Y, Lee T S, Pan C C, Huang A L, Chau L Y. Colocalization of iron and ceroid in human atherosclerotic lesions. Atherosclerosis. 1998;138:281-288).

In the setting of diabetes, there is a partial loss of function of Hp. It is for this reason that Hp 0 mice are relevant, namely, by allowing for the study of the importance of the loss of function of Hp. Renal and glomerular hypertrophy occurring in the Hp 0 mice is effectively reversed by an Hp 2 allele transgene in the absence of diabetes. This may be attributed to the ability of the Hp 2 protein to neutralize Hb and prevent Hb-induced oxidative damage. A hypothesis supporting the role of the Hp protein in regulating the development of renal disease via reducing Hb-induced oxidative stress is buttressed by the ability to inhibit renal hypertrophy in Hp 0 mice with antioxidant supplementation (vitamin E).

The increase in renal mass associated with the Hp 2 allele in the diabetic state is explained in one embodiment, by the synergy between Hp-type dependent differences in the clearance of Hp-Hb complexes and the inability of Hp to prevent glycosylated Hb-induced oxidation. In another embodiment, since the Hp-glycosylated Hb complex is oxidatively active, it is of heightened importance in the diabetic subject to clear the Hp-Hb complex as rapidly as possible. The Hp-2-2-Hb is cleared more slowly than Hp-1-1-Hb, thereby producing more oxidative stress in the tissues of Hp-2 mice and resulting in greater tissue damage in diabetic Hp-2 mice as compared to diabetic Hp 1 (wild type) mice.

Haptoglobin (Hp) is a highly conserved plasma glycoprotein and is the major protein that binds free hemoglobin (Hb) with a high avidity (kd, ˜1×10¹⁵ mol/L). Ischemia-reperfusion is associated with intravascular hemolysis and hemoglobin (Hb) release into the bloodstream. Extracorpuscular hemoglobin (Hb) is rapidly bound by Hp. The role of the Hp-Hb complex in modulating oxidative stress and inflammation after ischemia-reperfusion is Hp genotype dependent.

Vascular complications occur over time in diabetics, even though their blood sugar levels may be controlled by insulin or oral hypoglycaemics (blood glucose lowering) compounds. There are a number of vascular complications that diabetics are at risk of developing, those are diabetic retinopathy, diabetic cataracts and glaucoma, diabetic nephropathy, diabetic neuropathy, claudication, or gangrene, hyperlipidaemia or cardiovascular problems such as hypertension, cerebrovacular disease (stroke), atherosclerosis and coronary artery disease. In one embodiment, atherosclerosis may cause angina and heart attacks, and is twice as common in people with diabetes than in those without diabetes, affecting both men and women equally. In another embodiment, the vascular complication are exacerbated in subjects carrying the Hp-2 gene of haptoglobin and are encompassed in the scope of the methods of this invention.

In one embodiment, provided herein is a method for identifying in vivo a biological activity of a compound, wherein said biological activity is an oxidative stress, diabetes mellitus (DM), myocardial infract, vascular disease, nephropathy, retinopathy or cardiovascular disease.

Patients with diabetes exhibiting acute myocardial infarction (MI) have in one embodiment, an increased rate of death and heart failure. This poorer prognosis after MI in diabetic individuals is due in large part to an increase in MI size. Ischemia-reperfusion injury plays an important role in determining the amount of injury occurring with MI. Animal models of MI show in another embodiment, that the injury associated with ischemia-reperfusion is markedly exaggerated in the diabetic state. Increased oxidative stress characteristic of the diabetic state is compounded in one embodiment, during the ischemia-reperfusion process resulting in the increased generation of highly reactive oxygen species (ROS) which mediate in another embodiment, myocardial damage both directly and indirectly by promoting an exaggerated inflammatory reaction. Functional polymorphisms in genes that modulate oxidative stress and the inflammatory response are therefore of heightened importance in determining infarct size in the diabetic state. In one embodiment, biological compounds which exacerbate or in another embodiment ameliorate complications arising from MI in diabetic subjects can be analyzed according to certain embodiments of the methods of this invention.

Oxidative stress refers in one embodiment to a loss of redox homeostasis (imbalance) with an excess of reactive oxidative species (ROS) by the singular process of oxidation. Both redox and oxidative stress are associated in another embodiment, with an impairment of antioxidant defensive capacity as well as an overproduction of ROS.

The term “myocardial infract” or “MI” refers in another embodiment, to any amount of myocardial necrosis caused by ischemia. In one embodiment, an individual who was formerly diagnosed as having severe, stable or unstable angina pectoris can be diagnosed as having had a small MI. In another embodiment, the term “myocardial infract” refers to the death of a certain segment of the heart muscle (myocardium), which in one embodiment, is the result of a focal complete blockage in one of the main coronary arteries or a branch thereof.

Diabetic nephropathy refer in one embodiment, to any deleterious effect on kidney structure or function caused by diabetes mellitus. Diabetic nephropathy progresses in one embodiment in stages, the first being that characterized by microalbuminuria. This may progress in another embodiment, to macroalbuminuria, or overt nephropathy. In one embodiment, progressive renal functional decline characterized by decreased GFR results in clinical renal insufficiency and end-stage renal disease (ESRD).

Glucose combines in one embodiment, with many proteins in circulation and in tissues via a nonenzymatic, irreversible process to form advanced glycosylation end products (AGEs). The best known of these is glycosylated hemoglobin, a family of glucose-hemoglobin adducts. Hemoglobin A_{1c} (HbA_{1c}) is a specific member of this group and is useful in another embodiment, as an indicator of average glycemia during the months before measurement. Other AGEs are presumed to contribute to the complications of diabetes, such as glycosylated proteins of the basement membrane of the renal glomerulus. In one embodiment, candidate AGEs can be tested as biologically active agents according to the methods of this invention.

In one embodiment, retinal edema, hemorrhage, ischemia, microaneurysms, and neovascularization characterize diabetic retinopathy. In another embodiment advanced glycation end products (AGEs) cause the development of this complication. AGEs represent in one embodiment, an integrated measure of glucose exposure over time, are increased in diabetic retina, and correlate with the onset and severity of diabetic retinopathy. In one embodiment, specific high affinity receptors bind AGEs and lead to the downstream production of reactive oxygen intermediates (ROI). ROIs are correlated in another embodiment, with diabetic retinopathy and increase retinal VEGF expression. The inhibition of endogenous AGEs in diabetic animals prevents in another embodiment, vascular leakage and the development of acellular capillaries and microaneurysms in the retina. Compounds capable of inhibiting endogenous AGEs are screened and analyzed in one embodiment, according to the methods of the invention.

In many drug screening programs which test libraries of synthetic compounds and natural extracts, high throughput assays are used in one embodiment, in order to maximize the number of compounds screened in a given period of time. In another embodiment, assays performed in cell-free systems, such as may be derived with purified or semi-purified proteins, are often preferred as “primary” screens in that they can be generated to permit rapid development and relatively easy detection of an alteration in a molecular target which is mediated by a test modulating agent. In one embodiment, the effects of cellular toxicity or bioavailability of the test compound can be ignored in one embodiment, in the in vitro system, the assay instead being focused primarily on the effect of the drug on the molecular target as may be manifest in an alteration of binding affinity with upstream or downstream elements. In one embodiment the methods of the invention are used, with either the transgenic animals of the invention, their progeny or cell lines derived therefrom in a maner consistent with these screening programs.

Cardiovascular disease (CVD) is the most frequent, severe and costly complication of type 2 diabetes. It is the leading cause of death among patients with type 2 diabetes regardless of diabetes duration. In one embodiment, allelic polymorphism contributes to the phenotypic expression of CVD in diabetic subjects.

In the case of transgenic animals, the evaluation of the potentially useful compound for the treatment or prevention of pathology diabetic origin can be performed in one embodiment, by administration of the compound to be tested to said transgenic animal, at different doses, and evaluating the physiological response of the animal over time. In another embodiment, the administration of the compound to be assayed can be oral or parenteral, depending on the chemical nature of the compound to be evaluated. In one embodiment, it may be appropriate to administer the compound in question along with cofactors that enhance the effect of the compound.

In one embodiment, provided herein is a method for identifying in vivo a biological activity of a compound, wherein said biological activity is an oxidative stress, diabetes mellitus (DM), myocardial infract, vascular disease, nephropathy, retinopathy or cardiovascular disease, comprising ameliorating the abovementioned pathologies by administrating to said transgenic mouse and its progeny an effective amount of glutathione oxidase.

In another embodiment, glutathione peroxidase, is an important defense mechanism against myocardial ischemia-reperfusion injury, and is markedly decreased in one embodiment, in the cellular environment of DM. In vitro and in vivo studies with BXT-51072, a synthetic mimetic of glutathione peroxidase show in one embodiment, that glutathion peroxidase is capable of protecting cells against reactive oxygen species and in another embodiment, inhibit inflammation via action as an inhibitor of NF-κB activation.

In one embodiment, provided herein is a method for evaluating in a transgenic mouse the potential therapeutic effect of a compound for treating pathogenesis of a vascular disease in a human, which comprises: administering the compound to the transgenic mouse embodied herein, wherein said mouse exhibits at least one vascular disease which is a complication of diabetes mellitus (DM), myocardial infract, vascular disease, nephropathy, retinopathy or cardiovascular disease; and determining the therapeutic effect of the compound on the transgenic mouse.

In another embodiment, provided herein is a method for evaluating in a transgenic mouse the potential therapeutic effect of a compound for treating pathogenesis of a vascular disease in a human, by comparing in one embodiment the relative effect of the therapeutic effect of the compound, as compared with the therapeutic effects of glutathion peroxidase, other selenoorganic compounds, or in another embodiment, BXT-51072.

In one embodiment, provided herein is a method of making a transgenic mouse comprising: introducing into a mouse embryo a polynucleotide comprising a coding region which encodes Hp-2 gene product; transferring the embryo into a foster mother mouse; permitting the embryo to gestate; and selecting a transgenic mouse born to said foster mother mouse, wherein said transgenic mouse is characterized in that it has an increased probability of developing diabetes-related vascular complications when compared to a non-transgenic littermate.

In another embodiment, the introduction of the cDNA of the invention in the germ line of a non-human mammal is performed by means of microinjection of a linear DNA fragment that comprises the activatable gene operatively bound to the promoter that directs the expression of Hp-2 in fertilized oocytes of non-human mammals.

The fertilized oocytes can be isolated in one embodiment, by conventional methods; for example, provoking the ovulation of the female, either in response to copulation with a male in one embodiment, or by induction by treatment with the luteinising hormone in another embodiment. In general, a superovulation is induced in the females by hormonal action and they are crossed with males. After an appropriate period of time, the females are sacrificed in one embodiment, to isolate the fertilized oocytes from their oviducts, which are kept in another embodiment, in an appropriate culture medium. The fertilized oocytes can be recognised in one embodiment, under the microscope by the presence of pronuclei. The microinjection of the linear DNA fragment is performed in another embodiment, in the male pronucleus.

After the introduction of the linear DNA fragment that comprises the Hp-2 gene of the invention in fertilized oocytes, they are incubated in vitro for an appropriate period of time in one embodiment, or reimplanted in pseudopregnant wet nursing mothers (obtained by making female copulate with sterile males) in another embodiment. The implantation is performed by conventional methods, for example, anaesthetising the females and surgically inserting a sufficient number of embryos, for example, 10-20 embryos, in the oviducts of the pseudopregnant wet nursing mothers. Once gestation is over, some embryos will conclude the gestation and give rise to non-human transgenic mammals, which carry in one embodiment, the Hp-2 gene of the invention integrated into their genome and present in all the cells of the organism. In another embodiment, this progeny is the G0 generation and their individuals are the “transgenic founders”. The confirmation that an individual has incorporated the injected nuclear acid and is transgenic is obtained in one embodiment, by analysing the individuals of the progeny. To do this, the DNA is extracted from each individual animal, for example and in another embodiment, from the animal's tail or a blood sample in another embodiment, and analysed by conventional methods, such as, by polymerase chain reaction (PCR) using the specific initiators in one embodiment, or by Southern blot or Northern blot analysis using, for example, a probe that is complementary to, at least, a part of the transgene, or else by Western blot analysis using an antibody to the protein coded by the transgene in other embodiments. Other methods for evaluating the presence of the transgene include in other embodiments, appropriate biochemical assays, such as enzymatic and/or immunological assays, histological staining for particular markers, enzymatic activities, etc.

The progeny of a non-human transgenic mammal provided by this invention, such as the progeny of a transgenic mouse provided by this invention can be obtained in one embodiment, by copulation of the transgenic animal with an appropriate individual, or by in vitro fertilization of eggs and/or sperm of the transgenic animals. In another embodiment, the term “progeny” or “progeny of a non-human transgenic mammal” relates to all descendents of a previous generation of the non-human transgenic mammals originally transformed. The progeny can be analysed to detect the presence of the transgene by any of the aforementioned methods.

According to this aspect of the invention and in one embodiment, provided herein is a method of making a transgenic mouse comprising: introducing into a mouse embryo a polynucleotide comprising a coding region which encodes Hp-2 gene product; transferring the embryo into a foster mother mouse; permitting the embryo to gestate; and selecting a transgenic mouse born to said foster mother mouse, wherein following the selection of the transgenic mouse born to said foster mother mouse, transgenic male and female mice identified as such, from different parents are allowed to mate; permitting the embryos to gestate; and selecting a transgenic mouse born to the transgenic mother. In one embodiment, this process is repeated several generations.

In another embodiment, provided herein is a method of culturing transgenic cells comprising the steps of: providing the cell of any of the transgenic mice described herein; and culturing said cell under conditions that allow growth of said cell.

In the case of the cell lines of the invention, the evaluation of the potentially useful compound for the treatment or prevention of a pathology of diabetic origin can be performed in one embodiment, by adding the compound to be assayed to a cell culture medium for an appropriate period of time, at different concentrations, and evaluating the cellular response to the compound over time using appropriate biochemical or histological assays. In another embodiment, it may be necessary to add the compound in question to the cellular culture medium along with cofactors that enhance the effect of the compound.

In one embodiment, all the methods of the invention are carried out by contacting the cells obtained from the methods of the invention by the compounds contemplated by the invention. In another embodiment, when transgenic cells are used in the methods of the invention, indication of therapeutic effects will be analyzed on a cellular level, such as in another embodiment, by measuring concentration of VCAMs, ICAM's, selectins, ROS, or AGEs, VEGF, IL-10, Hb, Hb-Hp complex for example in other embodiments.

In one embodiment, these genetically modified mice serve as a platform on which pharmacological agents (iron chelation, antioxidants) designed to modify the risk of diabetic vascular disease as a function of Hp type may be tested. In man, there exists in one embodiment Hp genotype-specific differences in the clinical response to antioxidant therapy. A demonstration that these agents are effective in the Hp-modified mice in preventing vascular disease would provide in another embodiment, the impetus for pharmacogenomically designed prospective clinical trials with treatment dictated by the haptoglobin genotype.

The term “about” as used herein means in quantitative terms plus or minus 5%, or in another embodiment plus or minus 10%, or in another embodiment plus or minus 15%, or in another embodiment plus or minus 20%, around the specified term.

The following examples demonstrate certain embodiments of the invention. One of ordinary skill in the art will recognize the numerous modifications and variations that may be performed without altering the spirit or scope of the present invention. Such modifications and variations are believed to be encompassed within the scope of the invention. The examples do not in any way limit the invention.

### EXAMPLES

### Example 1

### Human HP 2 Mouse

Research design and methods

Hp 0, Hp 2, and wild-type mice

All protocols were approved by the Animal Care and Use Committee of the Technion Faculty of Medicine. C57Bl/6 mice were used as wild type (WT) (for haptoglobin). The generation and characterization of the haptoglobin knockout (Hp 0) mice propagated in a C57Bl/6 background has been previously described. The mouse endogenous haptoglobin gene is highly homologous to the human Hp 1 allele. The mouse haptoglobin gene and the human haptoglobin 1 allele both have 5 exons with identical exon-intron boundaries existing in mice and man. The Hp 2 allele exists only in man and contains 7 exons, arising from the Hp 1 allele early in human evolution by a partial intragenic duplication event. In summary, transgenic mice containing the human Hp 2 allele in a mixed genetic background were initially obtained and the Hp 2 allele was subsequently placed into a C57BL/6 background by 10 generations of backcrossing. These C57BL/6 Hp 2 transgenic mice were backcrossed with the Hp 0 mice to obtain mice with the murine Hp gene disrupted, but with a human Hp 2 allele transgene in a C57BL/6 background. Mice were fed a standard mice chow (Koffolk Ltd, Israel) with free access to water.

Construction of a Murine Hp 2 Allele.

One approach to model the Hp polymorphism in mice is to introduce the human Hp allele as a transgene (Hatada S, Kuziel W, Smithies O, Maeda N. The influence of chromosomal location on the expression of two transgenes in mice. J Biol Chem. 1999;274:948-955). Human Hp 2 transgenic mice in a Hp knockout background (Lim S K, Kim H, Lim S K, Ali A, Lim Y K, Wang Y, Chong S M, Costantini F, Baumman H. Increased susceptibility in Hp knockout mice during acute hemolysis. Blood. 1998;92:1870-1877) have been used to study mice expressing only the Hp 2 allelic protein product. However, these human Hp 2 allele transgenic mice have several serious shortcomings: (1) insertion of the human Hp 2 allele into the genome is random, i.e., not in the normal location of the murine Hp gene on chromosome 8. Therefore, the cell specific and inducible regulation of the human Hp 2 transgene is different from that of the endogenous murine Hp gene on chromosome 8; (2) it is difficult to study the heterozygote (Hp 2-1) and to differentiate between Hp 2 homozygote and Hp 2 hemizygote mice; (3) it is extremely cumbersome to backcross these mice with other transgenic mice in order to look at the interaction between the Hp genotype and other genes (i.e. Apo E) due to the need to select at three genetic loci; (4) the circulating levels of the protein product from the human Hp 2 allele are different from the levels of the wild type murine Hp 1 allele. This directly affects the polymeric distribution of the circulating Hp polymers found in the serum of these mice (Hatada S, Kuziel W, Smithies O, Maeda N. The influence of chromosomal location on the expression of two transgenes in mice. J Biol Chem. 1999;274:948-955.). We sought to overcome all of these problems by producing a transgenic mouse with a genetically engineered murine (as opposed to human) Hp 2 allele as described below.

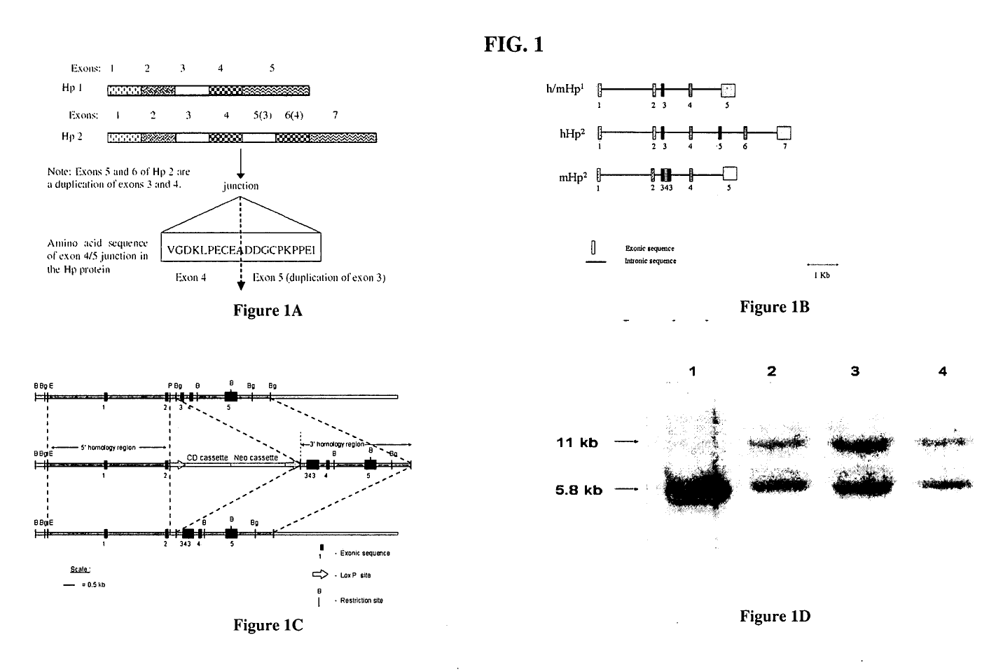

The human genomic locus as well as cDNAs encoding the Hp gene, both for the Hp 1 and Hp 2 alleles have been cloned and sequenced (Maeda N. Nucleotide sequence of the haptoglobin and haptoglobin-related gene pair. J Biol Chem 1985;260:6698-6709). The Hp 1 allele contains 5 exons and 4 introns. The Hp 2 allele contains 7 exons and 6 introns (FIG. 1B). The only difference between the two alleles is that the third and fourth exons of the Hp 1 allele have been duplicated in Hp 2 to give rise to exons 5 and 6 as well. Exon 5 in Hp 1 allele and exon 7 in the Hp 2 allele are identical. The reading frame of the duplicated region (exon 3 and 4) is maintained so the primary amino acid sequence produced by this duplicated region is a direct repeat of exons 3 and 4. Furthermore the translated in-frame amino acid sequence of exon 7 is the same as exon 5.

The genomic and cDNA sequence of mouse Hp is known (accession # M96827 C57BL/6J f) (Yang F, Linehan L A, Friedrichs W E, Lalley P A, Sakaguchi A Y, Bowman B H. Characterization of the mouse haptoglobin gene. Genomics 1993;18:374-380). The genomic structure of wild type murine Hp is remarkably similar to that of the human Hp 1 allele (FIG. 1B). There exist 5 exons and 4 introns in murine Hp. The nucleotide sequences at the intron-exon boundaries in mouse Hp and the human Hp 1 allele are 100% conserved. The overall amino acid homology between the murine and human Hp 1 alleles is over 80% (Maeda N. Nucleotide sequence of the haptoglobin and haptoglobinrelated gene pair. J Biol Chem 1985;260:6698-6709).

Because the nucleotide sequence at the intron-exon boundaries of the murine Hp 1 allele are conserved, it was possible to create a murine Hp 2 allele by duplicating murine exons 3 and 4. This duplication does not change the reading frame of sequences that come 3′ to the duplicated region allowing the sequence of the final exon (exon 7) to be read in frame unchanged from what occurs in the murine Hp 1 allele. Genomic mouse Hp DNA from the strain 129Sv obtained from a 129SvJ genomic library was kindly provided by Dr Sai-Kiang Lim and Dr Heinz Baumann. Our strategy to create a duplication of murine exons 3 and 4 was to modify murine exon 3 to become exon 343. In this strategy in the genomic murine Hp 2 allele there is no intron between the extra copy of exon 4 and the extra copy of exon 3. The intron normally occurring after exon 3 in the endogenous murine Hp 1 allele occurs after the 343 exon. The genomic structure of the murine Hp 2 allele is exon 1-intron 1-exon 2-intron 2 -exon3exon 4exon 3-intron 3-exon 4-intron 4-exon 5 (see FIG. 1B, murine Hp 2).

The genomic structure of the murine Hp 2 allele is different from the human Hp 2 allele in that there is no intron between the duplicated exons 3 and 4. However, in the mature mRNA (i.e., after the RNA has been spliced and intronic sequences removed) there will be no difference in the genetic organization of the murine and human Hp 2 alleles. The logic we used to generate a duplication and direct repeat of exons 3 and 4 in the murine Hp 1 allele can be explained as follows. Suppose exon 3 has sequence ABCDE and exon 4 has sequence FGHIJ. We cloned into the middle of exon 3 (at a restriction endonuclease site between AB and CDE) the sequence CDEFGHIJAB (i.e. 2nd half of exon 3, all of exon 4 and the 1st half of exon 3) thereby transforming exon 3 (ABCDE) into exon 343 (ABCDEFGHIJABCDE). Using this logic we generated a DNA fragment by RTPCR of Hp mRNA isolated from the human HepG2 hepatoma cell line with oligonucleotides 343sense (CGGGATCCATGACAGCTGCCCAAAGCCCCCAGAGA; SEQ ID NO:2) and 343 antisense CGGAATTCCAGCTGTCATCTGCCTCACATTCGGGGAGTTTCTC; SEQ ID NO:3). After digesting the fragment with PvuII we cloned it into the PvuII site of exon 3 of the murine Hp 1 allele to create a modified exon 3 with the sequence of exon3exon4exon3.

Once we replaced the murine 3 exon with a 343 exon, we proceeded to generate a targeting vector for transfection into embryonic stem (ES) cells. In designing targeting vectors for homologous recombination, it is critical that there is at least 2 kb of 100% homology sequences (regions identical between targeting vector and targeted gene) 5′ and 3′ to the targeted region. In our case the targeted region was exon 3 and the homology regions were murine genomic sequences located 5′ (5.6 kb) or 3′ (3.4 kb) to exon 3. A second feature of the targeting vector is a selectable marker, which can subsequently be removed. We used the neomycin antibiotic resistance gene (conferring resistance to G418) flanked by two lox P sites (allowing removal of the neo gene with the cre recombinase) for this purpose. We placed a cytosine deamninase (CD) gene casetted and a neo cassette in the intron between exon 2 and exon 343 bounded by 2 lox P sites using the cloning vector pTKLNCL (Thymidine kinase-LoxP-CD-Neo-LoxP) GB 135 (Levy J E, Jin O, Fujiwara Y, Kuo F, Andrews N C. Transferrin receptor is necessary for development of erythrocytes and the nervous system. Nature Genetics. 1999;21:396-399) (see FIG. 1C, for schematic picture of this construct after its successful integration showing the relationship between the wild type murine Hp 1 allele, and the targeting DNA after its integration both before (middle panel) and after (bottom panel) removal of the CD and Neo cassettes).

The targeting vector was linearized with Not I, transfected into 129O1a ES cells by electroporation (800 V, 3 uF) and individual clones selected with G4 18 (150 ug/ml). G418 resistant clones undergoing homologous recombination for the transfected sequences were identified by southern blot analysis of BamH1 digested DNA isolated from each clone using as a probe a 265 bp Bg/II-Bam HI fragment located outside (5′) of the 5′ homology region of the targeting vector. Southern blot using this probe yields a band of 5.8 kb in wild type mouse DNA (i.e. wild type murine Hp 1 allele) and 11 kb if the targeted Hp gene has undergone homologous recombination with the targeting vector (FIG. 1D): a southern blot of Bam H1 digested genomic DNA from wild-type mice showing a single 5.8 kb band recognized by the probe (lane 1). Lanes 2-4, a Southern blot of Bam H1 digested DNA from three different ES clones that have undergone successful gene targeting at one copy of chromosome 8 at the Hp locus (transgene integrated by homologous recombination at the Hp locus) demonstrate an additional band of 11 kb recognized by the probe.

Successfully targeted ES clones were then subjected to karyotope analysis and injected into 3.5 d post-coitum (dpc) C57BL/6J females to generate several chimeras. The chimeras were mated with C57BL/6J females to produce heterozygous Hp 2 mice that were then intercrossed to produce mice homozygous for the murine 2 allele. The CD and neo gene cassettes were deleted by crossing with EIIaCre mice overexpressing the cre recombinase in all tissues (provided by Heiner Westphal, National Institutes of Health). After the CD and neo gene casettes was deleted the only difference between the wild type murine Hp 1 allele and the murine Hp 2 allele which we created, other than exon 3, was in the intron between exons 2 and exons 3. In the murine Hp 1 allele the intron is 250 bp. In the murine Hp 2 gene a Pvu-Bgl fragment (100 bp) in the middle of this intron was deleted and additional sequences were inserted (vector sequences from pTKLNCL consisting of the Xho-LoxP and LoxP-Bam) thereby creating an intron between exons 2 and 343 in the murine Hp 2 allele of different length than the intron between exons 2 and 3 in the murine Hp 1 allele. These differences in the size of intron 2 have been exploited for Hp genotyping of the mice by PCR using oligonucleotides that bracket this intron. These oligonucleotides are: exon 2s AGCCCTGGGAGCTGTTGTCAC (SEQ ID NO:4; located in the coding sequence for exon 2) and 3r (located at the 3′ end of the intron between exon 2 and exon 3) TGGGTGCTCCGATGGCTCTCTG (SEQ ID NO:5). Oligonucleotides 2s and 3r yield a PCR product of 306 bp for the murine Hp 1 allele (83 bp from exon 2 and 223 from the intron) and 406 bp for the murine Hp 2 allele (83 bp from exon 2 and 323 from the intron). Mice having both bands are heterozygotes (haptoglobin 2-1).

Generation of a Murine Hp 2 Colony and Backcrossing with ApoE−/− Mice.

C57BL/6J mice containing the murine Hp 2 allele were backcrossed with C57Bl/6J mice for 10 generations. In order to assess the role of the Hp genotype in modulating aspects of atherosclerotic lesions we backcrossed these murine Hp 2 mice with C57Bl/6J ApoE−/− mice to generate C57Bl/6J ApoE−/−Hp2-2 mice. Genotyping at the Hp locus was achieved by analysis of tail DNA by PCR with oligos 2s and 3r as described above. Genotyping at the ApoE locus was performed by PCR based on the methodology recommended by the Jackson Laboratories using oligonucleotides IMR 0180 (GCCTAGCCGAGGGAGAGCCG; SEQ ID NO:6), IMR0181 (TGTGACTRGGGAGCTCTGCAGC; SEQ ID NO:7) and IMP0182 (GCCCGCCCCGACTGCATCT; SEQ ID NO:8). The ApoE wild type allele yields a band of 155bp, the targeted ApoE allele yields a band of 245 bp.

Mouse Studies (non-diabetic).

Total serum cholesterol (Roche), triglycerides (Roche) and HDL (Biosystems, Barcelona) were measured enzymatically. Serum Hp was measured based on the acid stable peroxidase activity of the Hp-Hb complex (Tridelta, Bray, UK).

The aortic arch was fixed in 4% formaldehyde, embedded in paraffin and sectioned using a Leica RM 2155 microtome. Total plaque area, lipid area, and minimum cap thickness were quantified as previously described (Moreno P R, Purushothaman K R, Fuster V, O'Connor W N. Intimomedial interface damage and adventitial inflammation is increased beneath disrupted atherosclerosis in the aorta: implications for plaque vulnerability. Circulation. 2002;105:2504-2511; Moreno P R, Lodder R A, Purushothaman K R, Charash W E, O'Connor W N, Muller J E. Detection of lipid pool, thin fibrous cap, and inflammatory cells in human aortic atherosclerotic plaques by near-infrared spectroscopy. Circulation. 2002;105:923-927).

Iron deposition. Iron deposition in the plaque was identified using Perl's stain (Asleh R, Guetta J, Kalet-Litman S, Miller-Lotan R, Levy A P. Haptoglobin genotype and diabetes dependent differences in iron mediated oxidative stress in vitro and in vivo. Circ Res. 2005;96:435-441) and quantified by measuring the percentage of plaque area staining black.

Lipid peroxidation and Ceroid. Lipid peroxidation was evaluated using the 4-hydroxynonenal (4-HNE) (Esterbauer H, Schaur R J, Zollner H. Chemistry and biochemistry of 4-hydroxynonenal, malondialdehyde, and related aldehydes. Free Rad Biol Med 1991;11:81-128) and the ceroid content of plaques (Kockx M M, Cromheeke K M, Knaapen M W M, Bosmans J M, De Meyer G R Y, Herman A G, Bult H. Phagocytosis and macrophage activation associated with hemorrhagic microvessels in human atherosclerosis. Arteroscl Thromb Vasc Biol 2003;23:440-446). As tissue fixation with formaldehyde is itself an oxidative process which may induce lipid peroxidation, this analysis was performed on frozen sections rather than on formaldehyde fixed tissue. 4-HNE, a lipid aldehyde, is a major end product of lipid peroxidation and is known to be increased in oxidative stress related disorders (Schaur R J, Zollner H, Esterbauer H. Biological effects of aldehydes with particular attention to 4-hydroxynenal and malondialdehyde. In: Vigo, Pelfrey C, ed. Membrane lipid peroxidation. Boca-Raton, Fla.: CRC Press; 1997:141-153). 4-HNE is especially reactive with Cys, His and Lys residues forming 4-HNE-protein adducts (Esterbauer H, Schaur R J, Zollner H. Chemistry and biochemistry of 4-hydroxynonenal, malondialdehyde, and related aldehydes. Free Rad Biol Med 1991;11:81-128) which can be identified by immunohistochemistry. Immunohistochemical detection of 4-HNE was performed using a rabbit polyclonal antibody to 4-HNE (Alexis Biochemicals) and a goat anti-rabbit antibody avidin biotin peroxidase complex (ABC kit, Vector Laboratories) according to manufacturer's instructions. The color reaction product was developed using 3,3′-diaminobenzidine tetrahydrochloride (DAB). Sections were counterstained with hematoxylin.

Ceroid is an insoluble complex of oxidized lipid and protein frequently identified in human atherosclerotic lesions. Ceroid is autoflourescent and was scored as the percentage of the total plaque area that was autofluorescent (Kockx M M, Cromheeke K M, Knaapen M W M, Bosmans J M, De Meyer G R Y, Herman A G, Bult H. Phagocytosis and macrophage activation associated with hemorrhagic microvessels in human atherosclerosis. Arteroscl Thromb Vasc Biol 2003;23:440-446). Ceroid was scored by two independent observers who were blinded to the Hp genotype of the specimen.

Macrophage accumulation. Immunohistochemical localization of macrophages was performed using formalin fixed, paraffin-embedded, 4-μm tissue sections on poly-lysine coated plus glass slides (Moreno P R, Purushothaman K R, Fuster V, O'Connor W N. Intimomedial interface damage and adventitial inflammation is increased beneath disrupted atherosclerosis in the aorta: implications for plaque vulnerability. Circulation. 2002; 105:2504-2511; Moreno P R, Lodder R A, Purushothaman K R, Charash W E, O'Connor W N, Muller J E. Detection of lipid pool, thin fibrous cap, and inflammatory cells in human aortic atherosclerotic plaques by near-infrared spectroscopy. Circulation. 2002;105:923-927). Tissue sections were deparaffinized and then pretreated with trypsin at 37° C. for 15 minutes. Sections were then incubated at 37° C. with 2% normal horse serum in Tris buffer to prevent non-specific antigen binding. Endogenous peroxidase activity was blocked with 10% H₂O₂ in methanol. Sections were incubated with mouse monoclonal anti-macrophage antibody clone SPM281 (Spring bioscience) for 60 minutes at 37° C. Secondary antibody avidin-biotin peroxidase complex was obtained from Vector Laboratories (ABC kit). Macrophages were identified using 3′,3′-diaminobenzidine tetrahydrochloride (DAB). Counterstaining was done using hematoxylin. Macrophages were counted manually in all plaques in the intima, media and adventitia (Moreno P R, Purushothaman K R, Fuster V, O'Connor W N. Intimomedial interface damage and adventitial inflammation is increased beneath disrupted atherosclerosis in the aorta: implications for plaque vulnerability. Circulation. 2002; 105:2504-2511; Moreno P R, Lodder R A, Purushothaman K R, Charash W E, O'Connor W N, Muller J E. Detection of lipid pool, thin fibrous cap, and inflammatory cells in human aortic atherosclerotic plaques by near-infrared spectroscopy. Circulation. 2002;105:923-927).

Statistical analysis. All results, with the exception of total plaque and lipid core area, are reported as the mean±SEM with differences between groups determined by a two-tailed t-test. Data for total plaque and lipid core area is reported as the 25th/50th/75th percentile with differences between groups determined by the Mann-Whitney test. A value of p≦0.05 was considered significant.

Results

Generation of a murine Hp 2 allele. The murine Hp 2 allele was engineered to have an intragenic duplication of exons 3 and 4, analogous to that found in the human Hp 2 allele (FIGS. 1B and 1C).

As shown in FIG. 1B, the human Hp 1 and Hp 2 alleles are located at chromosomal coordinates 16q22. The murine wild type Hp is a Hp 1 allele and is found on chromosome 8. A murine Hp 2 allele was created as described in this manuscript and inserted by homologous recombination at the wild type Hp locus replacing the murine Hp 1 allele. In the human Hp 2 allele, exons 5 and 6 represent a duplication of exons 3 and 4. The mouse Hp 1 allele has the identical intron-exon boundaries as the human Hp 1 allele and is 90% homologous at the amino acid level. The murine Hp 2 allele, constructed as described in the text, is similar to the human Hp 2 allele in that it has a direct repeat of exons 3 and 4. The exonic organization of the human and murine Hp 2 alleles are identical after RNA splicing has occurred.

In FIG. 1C, a fine map of the murine Hp locus before and after gene targeting is shown. Top. Genomic organization of the murine Hp 1 allele. B, Bam H1; Bg, Bgl II; E, EcoR1; P, PvuII. Middle. Genomic organization of the murine Hp 2 allele after successful gene targeting by homologous recombination. A targeting vector was constructed using the pTKLNCL GB 135 vector as a backbone. TKLNCL contains lox P sites (large arrow) bracketing the gene for cytosine deaminase (CD) and the neomycin (Neo) resistance gene. A 5.8 kb E-P fragment of the murine Hp 1 allele was cloned in the Kpn 1-Xho 1 site of TKLNCL 5′ to the neo cassette (5′ homology region) and a 3.4 kb BglII fragment of the murine Hp 1 allele was cloned in the Bam H1 site of TKLNCL 3′ to the neo cassette (3′ homology region). Exon 3 of the murine Hp 1 was reconstructed to be exon 343 as described in methods. The vector was linearized with Not 1 prior to transfection. Identification of G418 resistant ES clones which integrated the targeting vector at the Hp locus by homologous recombination was achieved by southern blot analysis of Bam H1 digested DNA from these clones using a 300 bp BamH1-Bgl II fragment (in blue) as probe. This probe hybridizes with a 5.8 kb Bam H1 fragment in wild type DNA (Hp 1) and with a 11 kb Bam H1 fragment in successfully targeted clones (Hp 2) (shown in FIG. 1 of on-line supplement). Bottom. Genomic organization of the murine Hp 2 allele after removal of the Neo and CD cassettes with cre recombinase.

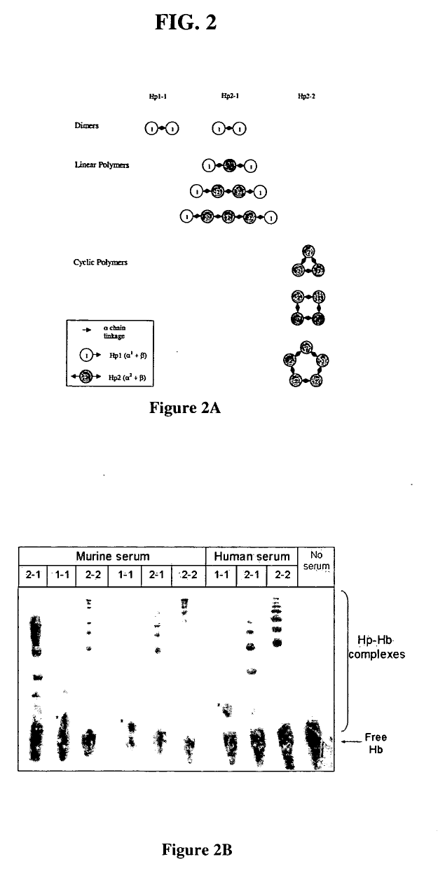

Once generated we used the murine Hp 2 allele to replace the normal mouse Hp 1 allele by homologous recombination. The shape and size of the murine Hp 2 allele protein product is similar to the human Hp 2 allele protein product. FIG. 2A shows schematically the difference as visualized by electron microscopy between the shape and size of Hp polymers found in humans with the Hp 1-1, 2-1 or 2-2 genotypes (Wejman J C, Hovsepian D, Wall J S, Hainfeld J F, Greer J. Structure and assembly of haptoglobin polymers by electron microscopy. J Mol Biol. 1984;174:343-368). Hp is synthesized as a single polypeptide which is proteolytically cleaved to give an alpha-chain (9 or 16 Kd derived from exons 1-4 or 1-6 for the 1 or 2 allele respectively) and a beta chain (45 Kd derived from exon 5 or exon 7 for the 1 or 2 allele respectively). The Hp alpha-beta monomer is covalently linked via disulfide bonds with other Hp monomers in a Hp genotype dependent fashion. This is because the cysteine residues responsible for Hp polymerization are present in the region of the Hp gene duplicated in the Hp 2 allele. A Hp monomer derived from the Hp 1 allele can be cross-linked with only one Hp monomer (it is monovalent) to form a Hp dimer. On the other hand, the Hp monomer derived from the Hp 2 allele is cross-linked with two Hp monomers (it is bivalent). In individuals with only the Hp 2 protein, the plasma Hp molecules are all cyclic polymers. In heterozygotes, Hp polymers are dimers, trimers and quatermers that are linear. These different polymeric structures can be easily visualized by taking advantage of the interaction of Hp with Hb and the peroxidase activity of Hb and Hb-Hp complexes. Electrophoresis on a non-denaturing polyacrylamide gel of Hb-enriched serum followed by immersal of the gel in 3,3′,5,5′-tetramethylbenzidine (forming a precipitate in the gel at the site of peroxidase activity) produces a signature banding pattern characteristic for each Hp genotype.8 In such gels, a single rapidly migrating band is seen in serum derived from Hp 1-1 individuals, corresponding to the Hp dimer, while more slowly migrating bands are seen in Hp 2-1 or Hp 2-2 individuals corresponding to the higher order linear and cyclic polymers present in these individuals (FIG. 2B). The cysteine residues of murine and human Hp are 100% conserved, and therefore the gene duplication event, which we have introduced in the murine Hp allele, would be predicted to result in a similar polymerization profile as the human Hp 2 allele. As demonstrated in FIG. 2B the banding pattern in a non-denaturing polyacrylamide gel of Hb-enriched serum from mice with the Hp 2 allele is remarkably similar to humans with the Hp 2 allele demonstrating that the gene duplication we have produced in the murine Hp 2 allele produces higher order Hp polymers similar to those seen in humans with the Hp 2 allele (FIG. 2B). Furthermore, the serum concentration of Hp protein was similar in mice with Hp 1-1 and Hp 2-2 genotypes (0.92±0.45 vs 1.10±0.37, p=0.66) and was similar to the Hp concentration reported for human serum (Langlois M R, Delanghe J R. Biological and clinical significance of haptoglobin polymorphism in humans. Clin Chem 1996;42:1589-1600).

FIG. 2 shows the size and shape of murine Hp 2 polymers are similar to human Hp 2 polymers. In FIG. 2A, a schematic illustration of the shape of Hp polymers in humans with the Hp 1-1, Hp 2-1 or Hp 2-2 genotypes is provided. The Hp monomer forms multimers whose stoichiometry is Hp genotype dependent. Multimerization is mediated by cysteine residue in exon 3 so that the Hp 1 allele protein product can combine with only one other monomer while the Hp 2 allele protein product combines with two other monomers. The structures shown have been verified by electron microscopy. FIG. 2B demonstrates that the polymer distribution in murine Hp 1-1, 2-1 and 2-2 mice is similar to that in humans with Hp 1- 1, 2-1 and 2-2. Shown is a polyacrylamide gel of serum samples from humans or mice with the indicated Hp genotypes. Samples were enriched with Hb and then electrophoresed on a non-denaturing polyacrylamide gel. Hp-Hb complexes were identified in the gel using a peroxidase sensitive reagent. A signature banding pattern is present for each Hp genotype. Note that higher molecular Hp-Hb complexes are absent in Hp 1-1 mice and that the distribution of the high molecular weight complexes in murine Hp 2-1 and Hp 2-2 mice is quite similar to that in humans with Hp 2-1 and Hp 2-2. Both the human Hp 1-1-Hb complex and the murine Hp 1-1-Hb complex are a single species (demarcated with an asterisk*) located just above the free Hb band.

Morphometric measurements of the atherosclerotic plaques. We characterized 18 plaques from 9 C57Bl6/6J ApoE−/−Hp1-1 mice and 15 plaques from 6 C57Bl6/6JApoE−/−Hp2-2 mice. There was no significant difference between the Hp 1-1 and Hp 2-2 mice with regard to age, weight, total serum cholesterol (432±67 mg/dl vs. 353±45 mg/dl, p=0.34), triglycerides (143±20 mg/dl vs. 101±12 mg/dl, p=0.15), or HDL cholesterol (22.3±4.6 mg/dl vs. 21.5±4.4 mg/dl, p=0.83). Fibrous cap thickness, plaque area and lipid core area in Hp 1-1 and Hp 2-2 mice are presented in Table 1. There was no significant difference in plaque or lipid core area between Hp 1-1 and Hp 2-2 mice. There was a non-significant trend showing decreased cap thickness in plaques from Hp 2-2 mice.

TABLE 1
Morphometric properties of plaques in Hp 1-1 and Hp 2-2 mice.

| | | Cap | Plaque | Lipid |
|---|---|---|---|---|
| | | thickness | area | core |
| Genotype | N | (um) | (um²) | (um²) |
| ApoE−/−Hp 1-1 | 18 | 19.1 ± 2.2 | 0.018/0.033/ | 0.006/0.017/ |
| | | | 0.144 | 0.035 |
| ApoE−/−Hp 2-2 | 15 | 15.0 ± 1.7 | 0.027/0.051/ | 0.008/0.022/ |
| | | | 0.084 | 0.035 |

N, total number of plaques analyzed. For cap thickness, the mean ± SME is shown. For plaque area and lipid core area the quartile values (25^{th}/50^{th}/75^{th} percentiles) are shown. There was no significant difference in cap thickness (p = 0.25), plaque area (p = 0.76) or lipid core area (p = 0.73) between Hp 1-1 and Hp 2-2 mice.

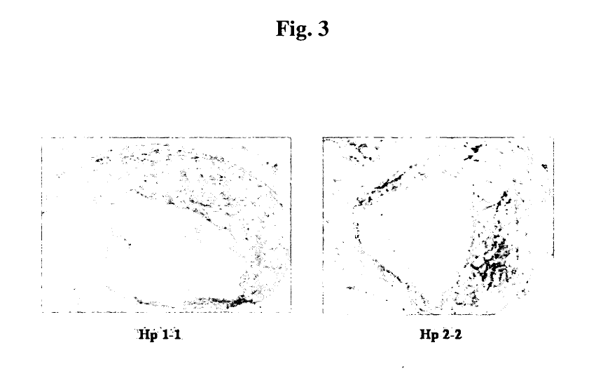

Increased iron deposition in Hp 2-2 plaques. Prior in vitro studies have suggested that hemoglobin released from microvascular hemorrhages within the plaque would be cleared more slowly in Hp 2-2 as compared to Hp 1-1 plaques.16 Consistent with this hypothesis, we found significantly increased iron staining, calculated as the percentage of the total plaque area, in Hp 2-2 plaques as compared to Hp 1-1 plaques. As shown in FIG. 3, intra-plaque iron is stained black (representative examples noted with arrows) with Perl's stain. The amount of iron staining in plaques from Hp 2-2 ApoE−/− mice was significantly greater than in Hp 1-1 ApoE−/− mice when scored as the percentage of the total plaque area (2.18±0.26% vs. 0.94±0.25%, n=10, p=0.008).

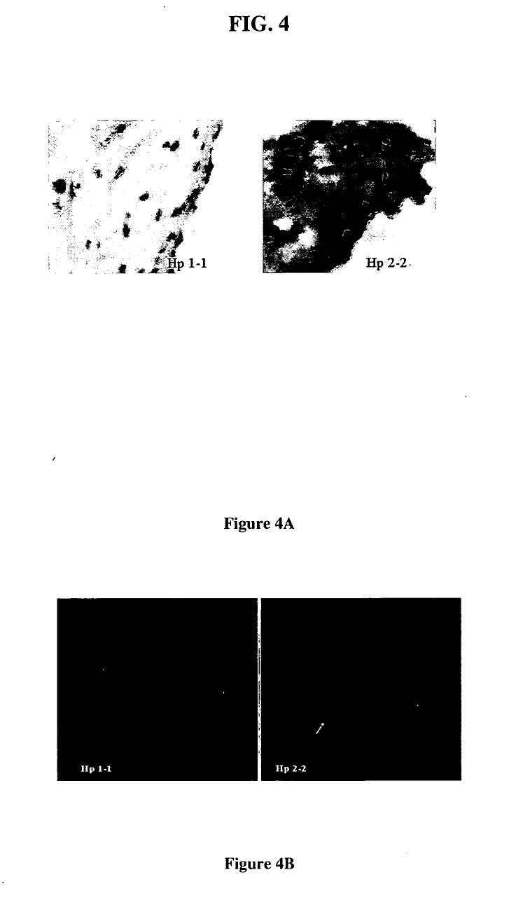

Increased lipid peroxidation in Hp 2-2 plaques. We assessed plaques for 4-HNE, a major end-product of lipid peroxidation, and ceroid, a mixture of autofluorescent oxidized lipid and protein. We found markedly greater 4-HNE (FIG. 4A) and ceroid (autofluorescence) (FIG. 4B) in the plaques of Hp 2-2 as compared to Hp 1-1 mice. The autofluorescent ceroid pigment (arrow) in the plaque was scored as the percentage of ceroid (autofluorescence) of the total plaque area. There was significantly more ceroid in Hp 2-2 plaques as compared to Hp 1-1 plaques (10.3±3.9% vs. 2.6±0.5% of total plaque area, n=8, p=0.05).

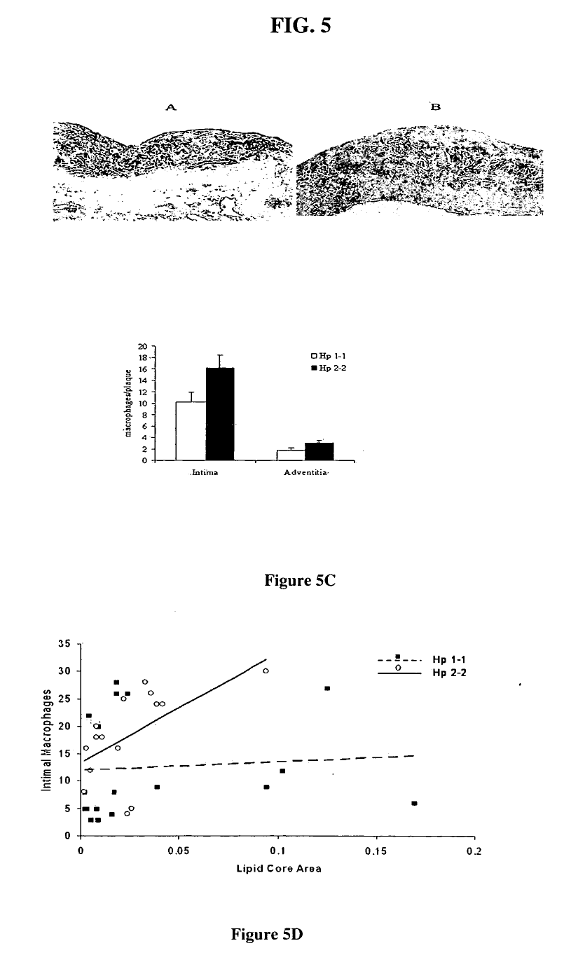

Increased macrophage accumulation in Hp 2-2 plaques. We found that in the intima and adventitia of atherosclerotic plaques from Hp 2-2 mice there were significantly more macrophages as compared to plaques from Hp 1-1 mice (FIG. 5). Macrophages were identified immunohistochemically as described in methods. Shown in (A) and (B) are representative plaques of similar size but with dramatically greater macrophage accumulation in Hp 2-2 Apo E−/− (A) as compared to Hp 1-1 ApoE−/− (B) mice. (C) Histogram of the mean±SEM of the number of macrophages in the intima and adventitia from all plaques (n=18 for Hp 1-1 and n=15 for Hp 2-2). There were significantly more macrophages in the intima (p=0.03) and adventitia (p=0.03) of plaques from Hp 2-2 as compared to Hp 1-1 mice. (D) Plot of the number of intimal macrophages vs. the lipid core area (um²) in plaques from Hp 1-1 ApoE−/− (n=18) and Hp 2-2 ApoE−/− (n=15) mice. There was a statistically significant correlation between the number of macrophages and the lipid core area in plaques from Hp 2-2 mice (correlation coefficient=0.57, p=0.01) but not in Hp 1-1 mice (correlation coefficient=0.08, p=0.38).

Correlation between lipid core size and inflammation in Hp 2-2 plaques but not in Hp 1-1 plaques. We found a significant correlation between the size of the lipid core and the number of intimal macrophages in plaques from Hp 2-2 mice (correlation coefficient r-0.57, p=0.01), while finding no correlation between the size of the lipid core and the number of macrophages in plaques from Hp 1-1 mice (correlation coefficient r=0.08, p=0.38) (FIG. 5D).

Mouse Studies (diabetic).

Increased renal hypertrophy in diabetic mice genetically modified at the haptoglobin locus. Diabetes was produced by intraperitoneal injection at 6 weeks of age with streptozotocin (Sigma Israel, Rehovot) at a concentration of 200 mg/kg dissolved in 50-mM citrate buffer pH 4.5. Glucose levels were monitored with a glucometer and a diagnostic kit from Sigma was used to measure HbAlc. Animals were sacrificed at 6 months of age. For these studies involving diabetes, a group of non-diabetic mice was followed in parallel so that the only difference between the groups was the presence or absence of diabetes.

Chronic supplementation with vitamin E. Wild-type or Hp 0 mice (n=5 in each group) were treated for 7 months with placebo (water) or vitamin E (Merck, racemic alpha-tocopherol acetate) at a concentration of 40 mg/kg/day administered daily in the drinking water from 4 weeks of age.

Preparation of renal tissue for morphometric and histochemical analysis. Mice were sacrificed with intraperitoneal injection of pentabarbitone sodium. Kidneys were excised and weighed and the half-middle portion was fixed in 4% buffered BP formaldehyde solution (Gadot, Netanya, Israel) and embedded in paraffin. For both the morphometric and histochemical analysis, there were four mice in each of the six groups (wild type, Hp 0, and Hp 2, with and without diabetes).

Morphometric analysis of glomeruli. Glomeruli in PAS-stained paraffin-embedded sections prepared as described above were analyzed using Image Pro software analysis. Measurements of glomerular dimensions (total glomerular area) were made on a minimum of 30 separate glomeruli for each kidney (n=4 for each group) and an average determined and used for analysis. One reader scored all glomeruli in the study and was blinded to the genotype of the mice.

Oxidative stress in kidney homogenates. The kidney was first diced into small pieces with a razor blade and then dounce-homogenized in 0.75 volumes of RIPA buffer (PBS containing 1% NP-40, 0.5% sodium deoxycholate, 0.1% sodium dodecyl sulfate, 2% beta-mercaptoethanol, 1 mM EDTA, 60 μg/mL aprotinin, 5 μg/mL leupeptin) at 4° C. The homogenate was then incubated at 4° C. for 30 min. PMSF (phenylmethylsulfonyl fluoride) was then added to 10 μg/mL and the homogenate again incubated for 30 min at 4° C. The homogenate was then centrifuged at 15 000^{a} g for 20 min at 4° C. The supernatant was aliquoted and stored at −70° C. until use. Protein concentrations in the supernatants were determined by Bradford protein assay. TBARS (thiobarbituric acid-reactive substances), a marker of oxidative stress, was determined in kidney homogenates (n=4 for each group) using a spectrophotometric assay, as previously described [24]. All values were normalized for protein and expressed in TBARS units (A532 OD units).

Statistical analysis. Values are reported as the mean±SEM. Comparisons between groups were performed using two-way ANOVA under a general linear model, with Hp phenotype as one factor and time, presence of diabetes, or treatment with vitamin E as the second factor. Pairwise comparisons were carried out using the Fisher's protected least significant difference (PLSD) test. A p-value of less than 0.05 was taken as being statistically significant. Statistical analyses were performed using the SPSS statistical software version 11.5 (Chicago, Ill.).

Renal and glomerular hypertrophy in Hp 0 mice and prevention with Hp 2 or vitamin E. Renal hypertrophy is a prominent feature of early diabetic renal disease both in mice and in man. Renal mass in the mice was determined with and without adjustment for total body weight (Table 2). In non-diabetic mice, there was no significant difference in young mice (3 months or less) between wild type, Hp 0, and Hp 2 mice. However, we found that renal mass in the non-diabetic mice was markedly increased in Hp 0 mice (6 months or more) relative to the WT and Hp 2 transgenic animals. There was no age-related difference between the renal mass of WT and Hp 2 transgenic animals in the absence of diabetes.

TABLE 2
Renal mass in non-diabetic mice and Hp genotype

| Hp | RM | RM/BM | RM | RM/BM | RM | RM/BM |
|---|---|---|---|---|---|---|
| genotype | 3-4 mo | 3-4 mo | 6-7 mo | 6-7 mo | 10-12 mo | 10-12 mo |
| WT | 0.32 ± 0.01 | 13.1 ± 0.55 | 0.34 ± 0.01 | 12.1 ± 0.34 | 0.34 ± 0.02 | 11.9 ± 0.56 |
| Hp 0 | 0.31 ± 0.01 | 12.2 ± 0.22 | 0.42 ± 0.02* | 13.6 ± 0.36* | 0.52 ± 0.01* | 16.3 ± 1.18* |
| Hp 2 | 0.31 ± 0.02 | 12.9 ± 0.63 | 0.32 ± 0.01 | 12.5 ± 0.55 | 0.34 ± 0.01 | 11.9 ± 0.42 |

Renal mass (RM) in mg with and without adjustment for total body mass (BM) in mg/g in Hp wild type (WT), Hp knockout (Hp 0), and Hp knock-in (Hp 2) mice segregated by age (3- to 4-month-, 6- to 7-month-, or 10- to 12-month-old mice). There was a significant increase in renal mass in Hp 0 animals by 6 to 7 months of age (*p<0.05 at 6 months and 10 months comparing WT to Hp 0 with and without adjustment for total body mass). Values are reported as the mean±SEM (n=5-20 for each group in 3- to 4-month range; n=10-12 for each group in 6- to 7-month range; n=4-5 for each group in 10- to 12-month range). There was no significant difference in body mass between the three different genotypes (WT, Hp 0, Hp 2).

Glomerular total area in non-diabetic mice (age 6 months) was examined using quantitative image analysis in the WT, Hp 0, and Hp 2 transgenic mice (Table 3). Two-way ANOVA using Hp phenotype as one factor and presence of diabetes as the second factor indicated that total glomerular area was higher in diabetic mice for all Hp phenotypes (p<0.0001 for the main effect of diabetes). However, we found a striking increase in the total glomerular area in the Hp 0 animals (both diabetic and non-diabetic) as compared to the WT and Hp 2 transgenic animals. There was no significant difference between glomerular area of WT and Hp 2 transgenic animals in the absence of diabetes. Similar results were obtained for measurements of the glomerular tuft area.

TABLE 3
Glomerular area and Hp genotype

| Hp genotype | Diabetes | Area | p |
|---|---|---|---|
| WT | | 4.2 ± 0.1 | |
| Hp 0 | | 5.1 ± 0.1* | 0.0001 < 0.24 |
| Hp 2 | | 4.4 ± 0.1 | |
| WT | + | 5.0 ± 0.1 | |
| Hp 0 | + | 5.3 ± 0.1 | 0.08 |
| Hp 2 | + | 4.9 ± 0.1 | 0.36 |

Glomerular area was measured using image pro software analysis in a cohort of animals 6 months old with and without diabetes and is reported in microns2^{a} 10-3. All values are expressed as the mean±SEM with a minimum of 4 animals from each group and 30 glomeruli measured for each animal. p-values are for the direct comparison between WT mice and Hp-modified mice with or without diabetes. There was a significant increase in glomerular area between Hp 0 mice without diabetes and WT mice without diabetes (p<0.0001). There was no significant difference between Hp 2 and WT mice in the presence or absence of diabetes.

Oxidative stress, as reflected in levels of malonaldehyde and 4-hydroxy-2(E)-nonenal, has previously been demonstrated to be increased in both the blood and tissues of Hp 0 mice. Two-way ANOVA using Hp phenotype as one factor and presence of diabetes as the second factor indicated that oxidative stress was higher in diabetic mice for all Hp phenotypes. However, in diabetic mice, we found a significant reduction in oxidative stress in renal tissue in Hp 2 mice compared to Hp 0 mice (TBARS expressed in A532 OD units of kidney extracts for 6-month-old diabetic mice was 0.39±0.01 for WT, 0.45±0.02 for Hp 0, and 0.37±0.02 for Hp 2, p=0.022 between WT and Hp 0 and p=0.55 between WT and Hp 2). These data suggested that reduction of increased oxidative stress found in Hp 0 mice by the Hp 2 transgene might have been of importance in preventing the development of renal hypertrophy from occurring in the Hp 2 mice. We therefore sought to prevent renal hypertrophy in Hp 0 mice with chronic antioxidant supplementation. Vitamin E or placebo was administered to wild type or Hp 0 animals for 7 months (the period of time sufficient to visualize differences between Hp 0 and wild-type mice with regard to renal hypertrophy). As demonstrated in Table 4, renal mass in Hp 0 animals receiving vitamin E was reduced compared to Hp 0 mice who did not receive vitamin E

TABLE 4
Inhibition of renal hypertrophy in Hp 0 mice with vitamin E

| Hp genotype | Vitamin E | RM/BM |
|---|---|---|
| WT | | 11.46 ± 0.16 |
| WT | + | 11.42 ± 0.98 |
| Hp 0 | | 12.42 ± 0.26* |
| Hp 0 | + | 11.01 ± 0.24** |

Wild-type (WT) or Hp knockout (Hp 0) mice were given 40 mg/kg/day vitamin E or placebo orally beginning at 4 weeks and sacrificed at 8 months of age. Values shown are for the mean±SEM of renal mass (RM) normalized for body weight (BM) (n=5 for each group) in mg/g.

* Indicates a statistically significant increase in renal mass in Hp 0 animals as compared to WT animals (p=0.02).

** Indicates a statistically significant decrease in renal mass in Hp 0 animals receiving vitamin E (p=0.003) as compared to Hp 0 mice who did not receive vitamin E

Renal mass and glomerular morphometric changes in diabetic animals. As described above, human Hp 2 allele transgene were found to be able to effectively replace the endogenous murine haptoglobin gene and restore normal kidney mass and glomerular size to Hp 0 mice. Differences between Hp 1 and Hp 2 mice would be expected to become manifested in the setting of diabetes due to the oxidative activity of glycosylated Hp-Hb complexes and the difference between the Hp proteins in clearing this species via the macrophage CD163 Hp-Hb scavenger receptor.

Accordingly, determination was made as to whether renal mass and glomerular hypertrophy would be greater in diabetic Hp 2 transgenic animals relative to diabetic WT animals (which contain only the murine haptoglobin gene, which is a class 1 allele). Beginning at 6 weeks of age, mice were made diabetic using streptozotocin and the consequences of the diabetes on renal mass and glomerular hypertrophy were assessed after 4 to 5 months of diabetic exposure. The severity of hyperglycemia produced was similar between mice with the different Hp genotypes. Both the diabetic WT and diabetic Hp 2 transgenic animals displayed an increase in renal mass and glomerular hypertrophy compared to their non-diabetic counterparts of similar age. However, renal mass in the diabetic Hp 2 transgenic animals was significantly greater than that seen in diabetic WT animals (Table 5). There was no difference in the amount of glomerular hypertrophy between Hp 2 diabetic mice and WT diabetic mice (Table 2).

TABLE 5
Renal mass, Hp genotype, and diabetes

| Hp genotype | RM/BM | p |
|---|---|---|
| WT | 16.3 ± 0.76 | |
| Hp 0 | 19.2 ± 0.88 | 0.04* |
| Hp 2 | 18.7 ± 0.53 | 0.02* |

Animals were made diabetic with streptozotocin beginning at 6 weeks of age and sacrificed at 6 months. Values shown are for the mean±SEM of renal mass (RM) normalized for body weight (BM) in mg/g in diabetic wild type (WT), diabetic Hp 0, and diabetic Hp 2 mice (n=minimum of 4 mice for each group)

* Indicates a significant increase in renal mass in diabetic Hp 2 mice and diabetic Hp 0 mice compared to diabetic WT mice.

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A transgenic mouse and progeny thereof whose genome comprises a nucleic acid encoding a humanized Hp-2 gene, wherein said humanized Hp 2 gene comprises the extracellular domain of a human Hp-2 gene, and said nucleic acid comprises exons 5 and 6 of a human Hp-2 gene, and exons 1,2 3, 4 and of a mouse or human Hp-1 gene.

2. The transgenic mouse of claim 1, wherein exons 5 and 6 of said human Hp-2 gene are a duplicate of exons 3 and 4 of said mouse or human Hp 1 gene respectively.

3. The transgenic mouse of claim 1, wherein said transgenic mouse exhibits, relative to a wild-type mouse, an increased sensitivity to vascular damage.

4. The transgenic mouse of claim 3, wherein said vascular damage is myocardial infract, vascular disease, nephropathy, retinopathy, neuropathy or cardiovascular disease.

5. A transgenic mouse and progeny thereof whose genome comprises a nucleic acid which does not encode murine Hp gene.

6. The transgenic mouse of claims 1 or 5, wherein said transgenic mouse is fertile and transmits said transgene to its offspring

7. A cell obtained from the transgenic mouse of claim 1 or 5.

8. A method for identifying in vivo a biological activity of a compound, said method comprising the steps of:
a. providing a transgenic mouse expressing humanized Hp-2 gene;
b. administering said compound to said mouse;
c. determining an expressed pathology of said mouse; and
d. identifying a in vivo biological activity of said compound.

9. The method of claim 8, wherein said biological activity is an oxidative stress, diabetes mellitus (DM), myocardial infract, vascular disease, nephropathy, retinopathy or cardiovascular disease.

10. The method of claim 9, comprising ameliorating said pathology by administrating to said transgenic mouse an effective amount of glutathione peroxidase or a mimetic thereof.

11. A method for evaluating in a transgenic mouse the potential therapeutic effect of a compound for treating pathogenesis of a vascular disease in a human, which comprises:
a. administering the compound to the transgenic mouse of claim 1, wherein said mouse exhibits at least one vascular disease which is diabetes mellitus (DM), myocardial infract, vascular disease, nephropathy, retinopathy or cardiovascular disease; and
b. determining the therapeutic effect of the compound on the transgenic mouse.

12. The method of claim 11, comprising comparing the therapeutic effect of the compound relative to the therapeutic effect of glutathione peroxidase.

13. A method of making a transgenic mouse comprising:
a. introducing into a mouse embryo a polynucleotide comprising a coding region which encodes Hp-2 gene product;
b. transferring the embryo into a foster mother mouse;
c. permitting the embryo to gestate; and
d. selecting a transgenic mouse born to said foster mother mouse,
wherein said transgenic mouse is characterized in that it has an increased probability of developing diabetes-related vascular complications when compared to a non-transgenic littennate.

14. The method of claim 13, wherein step d comprises mating two selected transgenic mice; permitting the embryos to gestate; and selecting a transgenic mouse born to a transgenic mother.

15. The method of claim 14, wherein the method is repeated for more than one generation.

16. A method of culturing transgenic cells comprising the steps of:
a providing the cell of claim 7; and
b. culturing said cell under conditions that allow growth of said cell.
